(19) United States
(12) Patent Application Publication
(10) Pub. No.: US 2005/0287071 A1
(43) Pub. Date: Dec. 29, 2005

(54) Formulation for a cement preparation as bone substitute

(75) Inventor: Robert Wenz, Wollstadt (DE)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Kyphon Inc., Sunnyvale, CA (US)

(21) Appl. No.: 11/141,769

(22) Filed: May 31, 2005

(30) Foreign Application Priority Data
Dec. 3, 2003 (WO) PCT/US03/38580
Dec. 3, 2002 (DE) 202 18 668.7

(51) Int. Cl. A61K 49/04; A61K 33/42

(52) U.S. Cl. 424/9.4; 424/602

(57) ABSTRACT

A bone cement comprises a powdery component including calcium phosphate and an aqueous liquid component. A radiopaque material is added to at least one of the powder and liquid components, and the components may be combined into a pasty substance for application to bone.

### CROSS-REFERENCE TO RELATED APPLICATIONS

This application is a continuation of International Application No. PCT/US2003/038580, filed Dec. 3, 2003, which claims the benefit under 35 U.S.C. §120.

### BACKGROUND OF THE INVENTION

1. Field of the Invention

The present invention relates to formulations for cement preparations as bone substitutes, comprising calcium phosphate as the main component and being admixed with water or aqueous liquids to obtain pasty or paste-like masses. The present invention also relates to processes for the preparation of phosphate cements, particularly apatite and struvite cements, from these formulations.

Calcium phosphate ceramics have been on the market as synthetic bone implants since the 1970's. Such ceramics, however, have fixed dimensions and have difficulty adapting bone defects which are usually irregularly shaped. A further disadvantage is that calcium phosphate ceramics are insufficiently resorbed into tissue. Such poor resorbtion results from the sintering preparation process which creates very dense structures.

The introduction of calcium phosphate cements in the 1980's was a significant advance over prior bone ceramics because they permitted complete filling of irregularly shaped bone defects and improved load transfer between bone and the implant. Such calcium phosphate cements are powders which are mixed with liquids to form a pasty substance which is capable of being introduced easily into the bone defects.

During solidification of such calcium phosphate cements, calcium phosphate precipitates because it is thermodynamically stable. Such precipitated calcium phosphate is degraded by body cells much better than are sintered materials since the solidified cements have a less dense structure than that of sintered materials. Such cements are described in U.S. Pat. No. 4,612,053; U.S. Pat. No. 5,149,368; U.S. Pat. No. 4,518,430; WO 96/14265; EP 1 296 909 A1, and EP 0 835 668 A1; which are incorporated herein by reference. Such cements are commercially available under trade names such as, BoneSource, Norian SRS, Biobon, Calcibon, and Cementek.

An object of the present invention is to provide a hardenable and resorbable bone or other cement preparation having improved safety and requiring less time for implanting, thus increasing the operator's freedom of choice when implanting or applying the cement preparation. It is a further object that the cement preparation have improved processing characteristics and provide increased strength of the hardened cement. It is a further object that the cement preparation have improved X-ray contrast.

2. Description of the Background Art

U.S. Pat. Nos. 4,612,053; 5,149,368; 4,518,430; PCT Publication WO 96/14265; and EP Publications EP 1 296 909 A1 and EP 0 835 668 A1, have been described above. Various calcium phosphate and other bone cements and fillers which incorporate radiopaque materials are described in U.S. Pat. Nos. 6,641,587; 6,599,520; 6,075,067; 6,375,659; WO 02/32474 (corresponding to U.S. Pat. No. 6,599,520); and WO 02/17801 (corresponding to U.S. Pat. No. 6,641,587). The full disclosures of each of the patents and published applications are incorporated herein by reference.

### BRIEF SUMMARY OF THE INVENTION

In a first aspect, the present invention provides a cement preparation useful as a bone substitute or filler and comprising calcium phosphate as the main component. The calcium phosphate component will typically be a powder and will comprise other materials. The powder will be adapted to mix with water or an aqueous liquid to form a pasty (viscous) substance for introduction to a bone defect or other bone target location. An additive enhancing X-ray contrast (radiopacity) is admixed with the cement preparation, preferably the dry powder calcium phosphate component, but in some cases with aqueous compounds prior to combining the components.

In a further aspect, the present invention provides a process for the preparation of a cement, comprising: mixing a calcium phosphate powder, preferably an apatite material with an additive enhancing the X-ray contrast and water or an aqueous component, and allowing the mixture to harden. The hardening allows formation of a cement, preferably an apatite cement, as the reaction product.

In particularly preferred embodiments, the cement preparation of the present invention comprises a mixture of salts of calcium salts, magnesium salts, and/or orthophosphates to be admixed with water and/or an aqueous liquid, wherein the additive for enhancing the X-ray contrast comprises at least one substance selected from the group consisting of: a barium salt; metals and inorganic and organic metal compounds, preferably metal oxides, wherein the metal is selected from the group consisting of iron, titanium, tantalum, gold, silver, rare earth elements, yttrium, ytterbium, zirconium, niobium, molybdenum, ruthenium, rhodium, palladium and tungsten; compounds of rare earth elements, preferably of gadolinium or cerium; inorganic or organic iodo compounds; and sintered hydroxyapatite and sintered tricalcium phosphate.

The cement preparations as defined above are particularly useful as bone substitutes. The term “bone substitute” includes bone replacement materials, bone implants, bone fillers, bone cements, bone adhesives, and the like. Such bone substitutes are useful for the treatment of bone defects and fractures and of disease conditions of the bone system, such as osteoporosis or cancer.

The compositions and methods of the present invention solves the problem of conventional calcium phosphate containing cements which, owing to the similar chemical structure of natural bones and the applied calcium phosphate containing cements, do not provide a satisfactory X-ray contrast. With the present invention, an additive possessing the property of enhancing or intensifying, relative to the formulation or composition lacking this additive, the X-ray contrast (radiopacity) significantly improves the capability of visualizing the treated location of the bone, which are often not directly observable, and allows the operating person to evaluate the progress and/or the result of the treatment in an X-ray image. The availability of images having higher X-ray contrast improves the safety of treatment. In addition the hardened cement of the invention also possesses good resorbtion characteristics.

With additives selected to improve X-ray contrast, which are optionally supplemented with auxiliary additives, the calcium phosphate powders combined with an aqueous liquid component according to the present invention provide a material having a paste-like (pasty) consistency having good flowability characteristics. Moreover, the materials form a hardened cement with minimum or no loss of product strength. The calcium phosphate compositions of the present invention are preferably free of plastics materials such as poly (methyl methacrylate) (PMMA), allowing the use of a variety of X-ray contrast enhancing additives at a wide range of concentrations because calcium phosphate cements do not harden through a polymeric chain reaction. Many X-ray additives would interfere with the polymerization reaction in such PMMA cements, particularly at high concentrations, and would diminish the strength of the hardened PMMA cement. Furthermore, the radiopacity enhanced bone cement preparations of the present invention which are hardened at the target site often have improved resorbtion characteristics. Thus, the compositions of the present invention may be optimized depending on the desired application. Depending on the chosen type of X-ray contrast enhancer, the composition may be formulated to be highly stable with radiopacity property for long term observations. Alternatively, the radiopacity may be diminished after the operation process in order to decrease the likelihood of body irritations due to the additive. Overall, the present invention provides a good combination of processability, hardening characteristics and enhanced visualization of the operation process.

### DETAILED DESCRIPTION OF THE INVENTION

The cement preparations of the present invention are based on a mixture of a powder component comprising mainly calcium phosphate, preferably at least about 50 percent by weight and more preferably at least about 65 percent by weight, and most preferably at least about 75 percent by weight calcium phosphate, to be mixed with an aqueous liquid component comprising water or, usually, an aqueous solution, for the hardening and the formation of a cement reaction product, typically used as a bone substitute. The basic powdery component preferably further comprises, in addition to calcium phosphate, orthophosphates or other salts of magnesium and optionally other metals. Ternary (tri-) calcium phosphate (TCP) of the α and/or β-type is particularly preferred. The liquid component comprises water and optionally further constituents, preferably salts and more preferably salts having a buffering effect, particularly sodium, potassium and/or ammonium salts of phosphoric acid in the primary and/or secondary basic form, or the corresponding salts of carbonic acid. The pH of the liquid component is suitably adjusted, for example within a range of about 5 to about 12, preferably from about 7 to about 12.

In order to provide a cement preparation with enhanced radiopacity, improved flowing characteristics, and improved mechanical properties (particularly improved strength upon hardening), the cement preparation preferably comprises a powdery component including calcium (Ca), magnesium (Mg), and orthophosphate (P). An additive which enhances X-ray contrast (also referred to herein as a radiopacity enhancer or a radiopaque material), which will be described in more detail below, is preferably combined in the powdery component and is advantageous in that the hardening characteristics and/or the mechanical properties is/are essentially not deteriorated. In addition, the hardened cement has particularly good resorbtion characteristics. In a further preferred embodiment, the basic cement preparation further comprises, either in the powdery component or preferably in the aqueous liquid component, an ammonium salt, in particular an ammonium phosphate salt such as (NH₄)₂HPO₄ and/or (NH₄)H₂PO₄. The molar ratios of Ca, Mg and P preferably lie in the range of 1.00<Ca/P<1.50 and 0<Mg/P<0.50. Particularly preferred formulations for cement preparations are the magnesium ammonium phosphate cement preparations disclosed in EP-A-1 296 909.

For the enhancement of X-ray contrast (radiopacity), in principle, all elements can be used for which the atomic number in the periodic table of elements is >20 and thereby higher than that of calcium. Depending on the element or the element compound used, it should be considered that the cement preparation provides a sufficiently enhanced radiopacity by means of selecting an appropriate type of radiopacity element or compound, and by using an appropriate amount, concentration and/or density of the radiopacity additive.

The additive for enhancing X-ray contrast (radiopacity) can be admixed with either the powdery component or the liquid component of the cement preparation, or both. The hardened reaction product obtained from the formulation or composition of the present invention may beneficially comprise crystalline compounds, particularly monocrystalline phosphate compounds, apatitic structures, hydroxyapatite structures, struvite cements, or tribarium phosphate.

The amount of the radiopacity enhancing additive, depending on the type the additive and the radiopacity enhancing effect on the hardened calcium containing phosphate cement, suitably constitutes a range between at least about 0.5 percent to about 25 percent by weight, preferably a range of at least about 3 percent to about 20 percent by weight, more preferably a range of at least about 5 percent to about 15 percent by weight, and most preferably at least about 10 percent by weight. The upper limit may be suitably selected depending on the type of the radiopacity enhancing additive and the intended application; the upper limit of the amount may, for example, be at about 50 percent by weight and more preferably at 25 percent by weight. The aforementioned preferred lower and upper limits of the amount of the radiopacity enhancing additive are particularly selected from the view point of a useful combination of significantly enhanced radiopacity while preserving or even improving the flowability characteristic of the cement preparations during operation when applying the cement preparation to the intended target. The above weight percentages are stated in relation to the weight of the powdery component. The aqueous liquid component is admixed with the powdery component in an amount in the range from about 0.1 to about 1.5 ml per mg powder, preferably from about 0.2 to about 0.65 ml liquid per mg powder.

In a preferred embodiment of the invention, an additive is used which at the same time maintains and preferably improves the flowability of the cement preparation being admixed to the pasty mass. Examples of such additives are strontium salts and particularly barium salts, used alone or in combination, e.g. a compound selected from the group consisting of strontium carbonate, strontium phosphate and barium sulfate. By the admixture of such compounds, the processability of the calcium phosphate containing cement preparations is additionally improved, in particular the injectability, because the admixture of these compounds significantly improves the flowability of the cement preparation being admixed with the liquid component. In addition, the pressure required for injecting the cement preparation is substantially decreased thereby. The decreased injection pressure is not only more comfortable and convenient for the operating person. Rather, the so-called “filter pressing” effect is minimized thereby as well. This effect is understood to mean the pressing out of liquid from the cement mixture made into a paste (cement paste) when pressure is applied thereon. This effect is very undesirable, because it results in an unacceptable solidification of the initially pasty cement mixture through liquid loss. In a particularly improved embodiment, strontium salt, e.g. strontium carbonate or strontium phosphate, is not used or not used alone and is replaced entirely or partly by barium salt such as barium sulfate, as the barium salt beneficially influences in addition the strength of the resulting hardened, calcium phosphate containing cement.

By the admixture of barium salts according to the present invention, a better cohesion is achieved while ensuring at the same time appropriate hardening characteristics. In addition, the mechanical properties, especially the strength of the hardened cement are significantly enhanced by barium salt, when combined with calcium phosphate and preferably additionally with magnesium phosphate in the cement preparation. Thus, by means of adding barium salts and particularly barium sulfate, tribarium phosphate, barium iodide, barium zirconate and barium wolframate, a combination of significantly improved radiopacity enhancement, improved processability and especially flowing characteristics, appropriate hardening characteristics as well as mechanical product properties is achieved.

Further particularly suitable additives for enhancing radiopacity which can be used according to the present invention include substances selected from the group of metals, inorganic metal compounds such as metal oxides, metal nitrides, metal carbides, metal silicids, metal halides, metal phosphates, metal gluconates, metal citrates, metal fumarates and metal sulfates, and metal-organic compounds, based on iron, titan, tantalum, gold, silver, rare earth elements, yttrium, ytterbium, molybdenum, zirconium, niobium, ruthenium, rhodium, palladium and tungsten as the metallic element. Preferred metallic elements are iron and rare earth elements (lanthanides), cerium and especially gadolinium being the rare earth element of choice. Particularly preferred substances of this type are iron compounds, especially iron phosphate, iron oxide, iron hydroxide or iron-compound with organic acids like iron citrate, because these can be incorporated into the hardened cement product in a stable manner and are particularly effective in not deteriorating the mechanical properties of the hardened product, and also tungsten salicylate and water soluble lanthanum or rare earth compounds such as lanthanum acetate, lanthanum nitrate, lanthanum sulfate, lanthanum ammonium nitrate, cerium citrate, cerium nitrate, cerium chloride, cerium ammonium sulfate, and especially gadolinium compounds which have been found to be advantageous, e.g. gadolinium fluorid, gadolinium chlorid, gadolinium chelates (e.g. gadolinium diethylenetriamino pentaacetate), Gadoteridol (available from Bristol-Myers Squibb).

Suitable metals and inorganic metal compounds such as oxides, nitrides, carbides, silicides and halides may be preferably added in a fine particulate form. The average particle size (d₅₀) of the particulate metal or inorganic metal compound in view of processability and especially flowing characteristics suitably lies in the range of about 0.1 nm to about 10 μm. The lower limit of the average particle size (d₅₀) preferably is about 5 nm, and the upper limit of the average particle size (d₅₀) is preferably about 1 μm, more preferably about 500 nm and further preferably about 100 nm. The fine particulate substance is preferably added to the powder component of the cement composition.

Also suitable are bromo compounds and particularly iodo compounds, preferably organic bromo and/or iodo compounds. As particularly preferred examples, water soluble compounds of ionic or non-ionic type are used according to the present invention, such as diatricoates, dioxitalamates, iopamidol, iohexol and ioxaglate or the like. Suitable examples among these iodo compounds can be found in section 35.2.2 of “Rote Liste” (Red List), ECV-Editio Cantor Verlag, Aulendorf (2003).

Additionally, sintered material may be used as the radiopacifier additive(s), preferably highly sintered materials. As preferred additives of this type, sintered hydroxyapatite and sintered tricalcium phosphate are preferred. The sintered material is suitably in a fine particulate form, and can be preferably added to the powder component of the cement composition, as described above in connection with the metal and inorganic metal compounds.

The aforementioned additive(s) for enhancing radiopacity may be added to the powdery component or to the liquid component of the cement preparation. Dry or fine particulate material is preferably added to the cement powder component, and water soluble compounds are suitably added to the liquid component of the cement preparation. Mixtures of the additives can be used as well.

Furthermore, depending on the purpose and the intended use, the additive for enhancing radiopacity may be capable of being stably incorporated into the reaction product of the cement preparation, for example iron compounds or compounds of other radiopacity enhancing metal elements like oxides, sulfates or phosphates. Alternatively, the additive for enhancing radiopacity is capable of being loosely incorporated but eliminated from, or leaked out of the hardened cement product after being applied to the desired target, in order to improve bio-compatibility and to minimize tissue irritation at the target site; for example, a water soluble additive for enhancing radiopacity may be selected, such as a water soluble iodo compound.

The formulations for cement preparations according to the present invention may contain other suitable additives. For example, the cement preparation of the present invention is particularly suitable as a carrier material for biologically and/or pharmaceutically active agents. For this purpose, the cement preparation may comprise in addition, in the powder and/or the liquid component, a pharmaceutically and/or biologically active substance, such as an antibiotic, a cytostatic agent, an analgesic agent, a disinfectant, a preservative, a growth factor, a proliferative factor, a protein or peptide, a biopolymer or the like, or a combination of the active substances mentioned. Particularly preferred active agents are selected from the group consisting of gentamycine, trombamycine, clindamycine, vancomycine, β-TGF or an analogue thereof, a bone morphogenic protein (BMP) series compound or the like, or a combination thereof.

Further additives include substances in the form of granular particles that are added to the powder component of the cement preparation of the present invention, wherein the granular particles of the substance possess the property of being water soluble in the liquid component of the cement preparation. Examples of such additives include salts, carbohydrates or sugars, and polymers capable of being degraded hydrolytically. These granular particles, being present in the powdery component in a suitable granular size of e.g. 10 to 300 μm, generate during the mixing and the hardening process a porous system which increases the surface area and accelerates the resorbtion performance of the reaction product.

The main application of calcium phosphate containing cement preparations according to the present invention resides in the augmentation (filling) of bone defects. In this respect, the filling of metaphysical depression fractures as well as vertebral bodies for stabilization in case of vertebroplasty or in case of compression fractures of osteoporotic vertebral bodies is of particular importance.

A high risk is incurred particularly in the case of the filling of vertebral bodies by calcium phosphate containing cements, if visualization control is insufficient. When introducing or applying the cement paste, material may extravasate from the vertebral body, e.g. may enter the spinal channel and may possibly lead to a compression of the spinal cord. The consequences thereof would be drastic for the patient, because paralysis conditions may occur.

In order to avoid this, the operation in the region of the vertebral bodies is carried out under image transfer control (X-ray control). Owing to the essentially equal X-ray density of cortical bones and of calcium phosphate containing cements, the image contrast would be very unsatisfactory if conventional cement mixtures were used. There is also often the interference by other bone structures. This particularly applies in the anterior/posterior beam path.

The inventive formulations having enhanced X-ray contrast bring about a substantial improvement when implanting the calcium phosphate containing cements according to the present invention, especially in the case of operations in the region of vertebral bodies. The operation can be carried out much faster, and the operation risk for the patient decreases drastically. In addition, excellent characteristics in terms of processability and product strength are feasible. The advantages mentioned here however likewise apply also to operations or treatments at other locations of the bones or the skeleton.

The calcium phosphate containing cement preparations may also be used for the filling of cavities or voids created by kyphoplasty. Kyphoplasty is a percutaneous technique involving the use of an expandable structure, such as a balloon catheter, to correct the kyphotic deformity related to vertebral body compression fractures. The methods and instruments suitable for such treatment are more fully described in U.S. Pat. Nos. 4,969,888, 5,108,404, 5,827,289, 5,972,015, 6,048,346, 6,066,154, 6,235,043, 6,241,734, 6,248,110, 6,280,456, 6,423,083, 6,440,138, 6,468,279, 6,575,919, 6,607,544, 6,613,054, 6,623,505, 6,641,587 and 6,645,213 each of which is incorporated herein by reference. The optimal use of the calcium phosphate containing cement preparation is in kyphoplasty, wherein a void or cavity of known size is created in bone, and a corresponding amount of a calcium phosphate containing cement preparation is introduced into the void.

### EXAMPLES

### Example 1

A cement powder composition with the following formulation components was provided, then thoroughly mixed with an aqueous 3.5 M (NH₄)₂HPO₄ solution (liquid component) to a pasty mass and subsequently allowed to harden:
65 g TCP (tricalcium phosphate)
12 g Mg₃(PO₄)₂
4 g MgHPO₄
3 g SrCO₃
2 g BaSO₄

The obtained hardened calcium phosphate cement was evaluated for its radiopacity as visualized on an X-ray film. A well recognizable X-ray contrast was obtained.

### Example 2

Example 1 was repeated except for increasing the amount of BaSO₄ to 8 g, as follows:
65 g TCP
12 g Mg₃(PO₄)₂
4 g MgHPO₄
3 g SrCO₃
8 g BaSO₄

An increased radiopacity due to the higher amount of BaSO₄ was obtained. The strength was measured after an incubation for 2 h of the hardened cement in an aqueous solution of 0.9 percent by weight NaCl at 37° C. A strength of 24.11 MPa was obtained.

### Example 3

Example 1 was repeated except for replacing the radiopacity enhancer by Ioxaglic, an ionic organic iodo compound, available from Guerbet GmbH (Sulzbach-Taunus, Germany)
65 g TCP
12 g Mg₃(PO₄)₂
4 g MgHPO₄
3 g SrCO₃
2 g Ioxaglic

### Example 4

Example 3 was repeated except for replacing the radiopacity enhancer loxaglic by Iobitridol, a nonionic organic iodo compound, available from Guerbet GmbH (Sulzbach-Taunus, Germany).
65 g TCP
12 g Mg₃(PO₄)₂
4 g MgHPO₄
3 g SrCO₃
2 g Iobitridol

Result: A similar radiopacity as in Example 3 was obtained. In comparison, Iobitridol provides a cement composition having an improved processability, because the compound is admixed with the other components in a beneficial and more homogeneous manner. The X-ray contrast is improved in comparison with the addition of the same amount of BaSO₄ according to Example 2.

### Example 5

Example 4 was repeated except for increasing the amount of Iobitridol to 8 g, as follows:
65 g TCP
12 g Mg₃(PO₄)₂
4 g MgHPO₄
3 g SrCO₃
8 g Iobitridol

Result: The X-ray contrast is remarkably higher (darker level of the developed X-ray image) than when adding 8 g BaSO₄. The strength was measured after an incubation for 2 h of the hardened cement in an aqueous solution of 0.9 percent by weight NaCl at 37° C., and a value of 17.5 Mpa was obtained.

### Examples 6 and 7

Examples 3 and 4 were repeated except for using an amount of Ioxaglic or Iobitridol of 6 g, as follows:

| | |
|---|---|
| 65 g TCP | 65 g TCP |
| 12 g Mg₃(PO₄)₂ | 12 g Mg₃(PO₄)₂ |
| 4 g MgHPO₄ | 4 g MgHPO₄ |
| 3 g SrCO₃ | 3 g SrCO₃ |
| 6 g Ioxaglic | 6 g Iobitridol |

Result: The wettablility of the powder component of the cement composition with the admixed liquid component (3.5 M (NH₄)₂HPO₄ solution) is better when adding Iobitridol than when adding Ioxaglic.

### Example 8

Example 1 was repeated except for replacing Ba₂SO₄ by another radiopacity enhancer, ammonium cer(IV)sulfat-dihydrate, as follows:
65 g TCP
12 g Mg₃(PO₄)₂
4 g MgHPO₄
3 g SrCO₃
5 g ammoniumcer(IV)sulfat-dihydrate

Result: The contrast corresponds to that of adding 9 g BaSO₄.

### Example 9

Further experiments have been carried out by adding various amounts of BaSO₄ or SrCO₃ to the cement powder. Best results in terms of homogeneous and more efficient admixing performance and strength of the hardened product have been achieved by adding about 10 to about 15 percent by weight BaSO₄.

The results of cement formulations comprising BaSO₄ are beneficially reproducible with respect to the strength characteristics, because the material has a lower tendency of clotting during the milling process.

### Example 10

All Examples described above have also been carried out with the difference that the formulations did not contain SrCO₃, i.e. using the following cement powder preparations with the liquid component:

65 g TCP, 12 g Mg₃(PO₄)₂, 4 g MgHPO₄, wherein the radiopacity enhancers according to the Examples 1 to 9 have been added. There was no discernible effect on radiopacity.

### Example 11

Example 1 was repeated except for replacing the radiopacity enhancer by gold dust, being added in the form of fine metal particles.
65 g TCP
12 g Mg₃(PO₄)₂
4 g MgHPO₄
3 g SrCO₃
2 g gold dust

Result: A good and homogeneous admixing performance is enabled. The X-ray contrast is equivalent to the addition of 15 g BaSO₄ instead of 2 g gold dust into the same cement preparation.

### Examples 12 and 13

Example 1 was repeated except for replacing the radiopacity enhancer by Fe(II) or Fe(III) oxide, being added as a metal oxide, as follows:

| | |
|---|---|
| 65 g TCP | 65 g TCP |
| 12 g Mg₃(PO₄)₂ | 12 g Mg₃(PO₄)₂ |
| 4 g MgHPO₄ | 4 g MgHPO₄ |
| 3 g SrCO₃ | 3 g SrCO₃ |
| 5 g Fe0 | 5 g Fe₂O₃ |

Result: The addition of 5g Fe₂O₃ results in a particular high X-ray contrast. The final strength of the hardened cement is not deteriorated.

While the above preferred embodiments and examples have been described for illustrating the present invention, it is to be noted that the features, examples and embodiments of the invention are not limited thereto but comprises equivalents, modifications and combinations thereof. In particular, the present invention shall not be construed in a way narrower than expressed by the scope and the spirit of the appended claims.

1. A cement preparation useful as bone substitute comprises a calcium phosphate powder which may be combined with an aqueous liquid, wherein the at least one of the powder or aqueous liquid is admixed with a radiopaque material prior to combination into the cement.

2. The preparation according to claim 1, further comprising a flowability enhancing additive for improving the flowability of the cement preparation after being combined with the liquid.

3. The preparation according to claim 1, wherein the admixed additive is combined with the calcium phosphate containing powder composition, whereby hardening characteristics of the cement preparation and/or mechanical properties of the hardened product are essentially not deteriorated.

4. The preparation according to claim 1, wherein the radiopaque material comprises a barium salt.

5. The preparation according to claim 4, wherein the barium salt is selected from the group consisting of barium sulfate, tribarium phosphate, barium jodide, barium zirconate and barium wolframate.

6. The preparation according to claim 1, wherein at least one of a metal, an inorganic metal compound, and a metal organic compound is admixed as the additive, wherein the metal or the metal compound is based on any metal element selected from the group consisting of iron, titanium, tantalum, gold, silver, rare earth elements, yttrium, ytterbium, zirconium, niobium, molybdenum, ruthenium, rhodium, palladium and tungsten.

7. The preparation according to claim 6, wherein the metal compound is selected from the group consisting of iron phosphate, iron oxide, iron hydroxide, and iron compounds with organic acids.

8. The preparation according to claim 1, wherein the additive comprises a compound of an element selected from the group of rare earth elements.

9. The preparation according to claim 1, wherein an iodo compound is admixed as the additive.

10. The preparation according to claim 9, wherein the iodo compound is an organic iodo compound of ionic or non-ionic type, preferably diatrizoate, ioxidalamate, iotamidol, iohexol or ioxaglate or a mixture thereof.

11. The preparation according to claim 1, wherein the admixed additive is a water soluble compound being admixed to the aqueous liquid component.

12. The preparation according to claim 11, wherein the metal or the metal oxide is admixed as the additive in a fine particulate form.

13. The preparation according to claim 1, wherein a sintered material, preferably sintered hydroxyapatite and/or sintered tricalcium phosphate, is admixed as the additive.

14. The preparation according to claim 1, wherein the additive for enhancing X-ray contrast is admixed in an amount of about 2.5 percent by weight to about 50 percent by weight relative to the total amount of the cement preparation.

15. The preparation according to claim 1, wherein the cement preparation comprises calcium, magnesium, and orthophosphates and optionally other salts of orthophosphoric acid as components of the composition.

16. The preparation according to claim 15, wherein the cement preparation further comprises an ammonium salt, preferably in the aqueous liquid component in the form of ammonium phosphate and/or ammonium hydrogen phosphate.

17. The preparation according to claim 1, wherein the cement preparation with the aqueous liquid is capable of providing a hardened cement which comprises at least one of the structural components selected from the group consisting of an apatite structure, a hydroxyapatite structure, a struvite structure and a tribarium phosphate structure.

18. A cement preparation comprising a powdery component including an admixture of salts of calcium, magnesium, and orthophosphate to be admixed an aqueous liquid component, wherein the cement preparation further comprises at least one additive for enhancing the X-ray contrast selected from the group consisting of:
a barium salt;
metals and inorganic and organic metal compounds, preferably metal oxides, wherein the metal is selected from the group consisting of iron, titanium, tantalum, gold, silver, rare earth elements, yttrium, ytterbium, zirconium, niobium, molybdenum, ruthenium, rhodium, palladium and tungsten;
compounds of rare earth elements, preferably of gadolinium or cerium;
inorganic or organic iodo compounds; and
sintered hydroxyapatite and sintered tricalcium phosphate.

19. The cement preparation according to claim 18, wherein the additive is admixed as a fine particulate form to the powdery component of the cement preparation.

20. The cement preparation according to claim 18, wherein the additive is a water soluble compound and is admixed to the water or aqueous liquid component of the cement preparation.

21. The cement preparation according to claim 18, further comprising a strontium salt in the powders component.

22. The cement preparation according to claim 18, wherein the additive is a barium salt selected from the group consisting of barium sulfate, tribarium phosphate, barium jodide, barium zirconate and barium wolframate, and/or that the metal compound is selected from the group consisting of iron phosphate, iron oxide, iron hydroxide, and iron compounds with organic acids.

23. The preparation according to claim 1 or the cement preparation according to claim 18, wherein a pharmaceutically and/or biologically active substance is further admixed to the cement preparation.

24. A hardened bone substitute material as obtained from the preparation according to claim 1 or the cement preparation according to claim 18.

25. The hardened bone substitute material according to claim 24, wherein the hardened cement comprises at least one of the structural components selected from the group consisting of an apatite structure, a hydroxyapatite structure, a struvite structure and a tribarium phosphate structure.

26. A use of the hardened bone substitute material according to claim 24 as bone implant, bone filler, bone cement or bone adhesive or as a therapeutic agent for the treatment of bone defects or osteoporosis.

27. A method for the treatment of bone defects or for the treatment of disease condition of the bone system, comprising a step of applying a formulation according to claim 1 or a cement preparation according to claim 18 to the defect site of a bone or a portion of the skeleton of a patient in need of such treatment.

28. The method according to claim 27, wherein the bone defect is applied to a vertebral body of the patient.

29. A process for the formation of a calcium phosphate containing cement, comprising the steps of:
mixing the powdery and liquid components of the formulation as defined in claim 1 or of the cement preparation as defined in claim 18; and
allowing the obtained mixture to harden.
